(19) United States
(12) Patent Application Publication
(10) Pub. No.: US 2026/0325921 A1
(43) Pub. Date: Oct. 22, 2026

(54) EXERCISE SUPPORT METHOD, EXERCISE SUPPORT DEVICE, EXERCISE SUPPORT SYSTEM, AND NON-TRANSITORY COMPUTER READABLE MEDIUM

(71) Applicant: CASIO COMPUTER CO., LTD., Tokyo (JP)

(72) Inventor: Nobuyoshi NISHIZAKA, Tokyo (JP)

(73) Assignee: CASIO COMPUTER CO., LTD., Tokyo (JP)

(21) Appl. No.: 19/553,215

(22) Filed: Feb. 28, 2026

(30) Foreign Application Priority Data
Mar. 19, 2025 (JP) 2025-044647

(51) Int. Cl. A63B 24/00; G16H 20/30

(52) U.S. Cl. CPC A63B 24/0075; G16H 20/30; A63B 2024/0068; A63B 2230/62

(57) ABSTRACT

An exercise support method includes: acquiring an indicator value related to body movement information of a user during exercise; causing an outputter to output advice information on the exercise of the user, based on the acquired indicator value; and determining whether the indicator value acquired after outputting the advice information has improved over the indicator value acquired before outputting the advice information. When it is determined that the indicator value has not improved even though the outputter has outputted the advice information, the outputter is caused to output advice information that includes a different expression from the advice information that has already been outputted and with which the indicator value has not improved.

### CROSS-REFERENCE TO RELATED APPLICATION

This application claims the benefit of Japanese Patent Application No. 2025-44647, filed on Mar. 19, 2025, the entire contents of which are incorporated herein by reference.

### BACKGROUND OF THE INVENTION

### Technical Field

The present invention relates to an exercise support method, an exercise support device, an exercise support system, and a non-transitory computer readable medium.

### Description of the Related Art

There is known a technique of detecting exercise condition of a target (e.g., a test subject) using a wearable device worn on the target and evaluating the exercise condition based on the analysis results of the detected exercise condition. For example, Japanese Unexamined Patent Application Publication No. 2020-150987 describes a technique of presenting advice information on the form of a certain exercise to a target who has performed the certain exercise, based on the analysis results of the exercise condition of the target.

### SUMMARY OF THE INVENTION

One of the advantages of the present invention is to provide an exercise support method, an exercise support device, an exercise support system, and a non-transitory computer readable medium for efficiently improving indicator values regarding body movement information of a user during exercise.

According to the present disclosure, an exercise support method includes: acquiring an indicator value related to body movement information of a user during exercise; causing an outputter to output advice information on the exercise of the user, based on the acquired indicator value; and determining whether the indicator value acquired after outputting the advice information has improved over the indicator value acquired before outputting the advice information, wherein in a case where it is determined that the indicator value has not improved even though the outputter has outputted the advice information, the outputter is caused to output advice information that includes a different expression from the advice information that has already been outputted and with which the indicator value has not improved.

### BRIEF DESCRIPTION OF DRAWINGS

FIG. 1 is a block diagram showing an exercise support system of an embodiment of the present disclosure.

FIG. 2 is a block diagram showing a functional configuration of a sensor device.

FIG. 3 is a block diagram showing a functional configuration of an information terminal.

FIG. 4 is a block diagram showing a functional configuration of wireless earphones.

FIG. 5 is a block diagram showing a functional configuration of a server.

FIG. 6 is a flowchart showing a control procedure in a form improvement advice process.

FIG. 7 shows an example of scoring results of form items.

### DETAILED DESCRIPTION

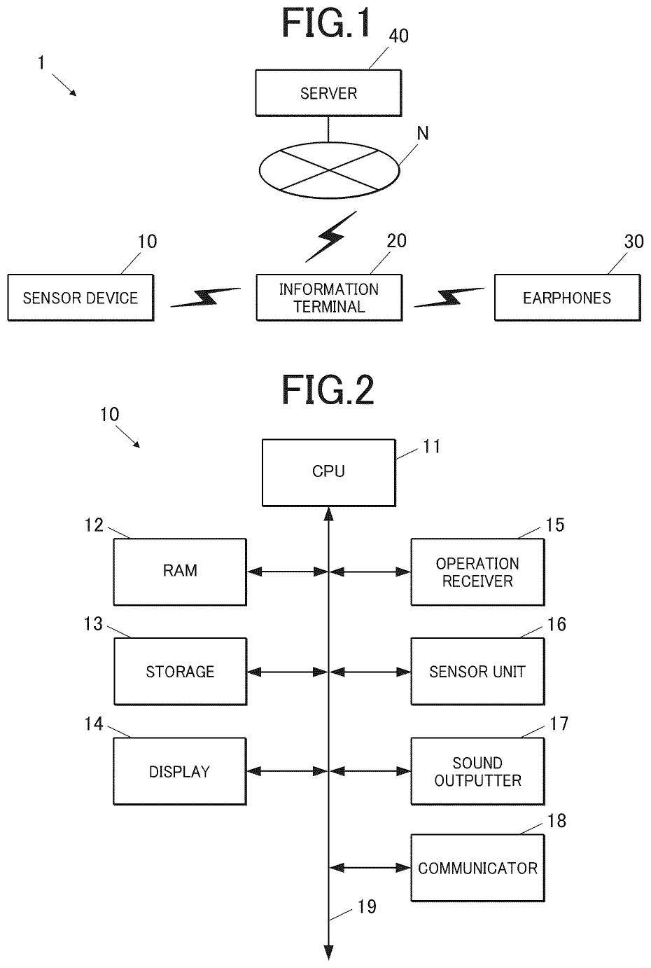

Hereinafter, an embodiment of the present disclosure will be described with reference to the drawings. First, the configuration of an exercise support system as the embodiment of the present disclosure is described with reference to FIG. 1. The exercise support system provides a user with advice information for improving running form during running. As shown in FIG. 1, the exercise support system 1 includes a sensor device 10, an information terminal 20 as an exercise support device, earphones 30, and a server 40.

The sensor device 10 is a wearable terminal that is worn on the body (e.g., waist) of the user of the exercise support system 1 when running. The information terminal 20 is a portable terminal that is carried and used by the user when running. The earphones 30 are an ear device set worn on the ears of the user when running. Herein, the earphones 30 are wireless earphones. The server 40 is a cloud server that generates text data of advice information and provides the advice information to the information terminal 20. The information terminal 20 is communicatively connected to the sensor device 10 and the earphones 30 via Bluetooth (registered trademark) Low Energy (BLE) or Wi-Fi (registered trademark). The information terminal 20 and the server 40 are communicatively connected via a communication network N. The communication network N is a network that relays communications between the information terminal 20 and the server 40. The communication network N is the Internet, for example.

Next, a functional configuration of the sensor device 10 is explained with reference to FIG. 2. As shown in FIG. 2, the sensor device 10 includes a central processing unit (CPU) 11 (at least one processor), a random access memory (RAM) 12, a storage 13, a display 14, an operation receiver 15, a sensor unit 16, a sound outputter 17, and a communicator 18. These parts of the sensor device 10 are connected via a bus 19. The CPU 11 controls each part of the sensor device 10. The CPU 11 reads the designated program from system programs and application programs stored in the storage 13, loads the program in the RAM 12, and executes various processes in cooperation with the program. The CPU 11 also includes a non-illustrated timekeeping circuit and acquires the current time as timed by the timekeeping circuit. The RAM 12 is a volatile memory and forms a work area that temporarily stores various data and programs. The storage 13 is a non-transitory computer readable medium and includes a flash memory and/or an electrically erasable programmable ROM (EEPROM), for example. The storage 13 stores system programs and application programs executed by the CPU 11, as well as data necessary for the execution of these programs.

The display 14 includes LED lamps and can display the ON/OFF status of the power supply, the data acquisition status (e.g., whether data is being acquired or not), the data transmission status (e.g., whether data is being transmitted or not), and the ON/OFF status of the GPS receiver, for example. The operation receiver 15 includes a power button (not shown) to turn the power on and off. The CPU 11 controls each part of the sensor device 10, based on the instructions from the operation receiver 15. The sound outputter 17 includes a DA converter, an amplifier, and a speaker. When outputting sound, the sound outputter 17 converts sound data into analog sound data and outputs the analog sound data from the speaker. The communicator 18 performs wireless data communication with the second communicator 28 of the information terminal 20 in accordance with the communication standards related to wireless communication by BLE or Wi-Fi.

The sensor unit 16 includes a tri-axial acceleration sensor, a gyro sensor, a geomagnetic sensor, and a GPS receiver. The sensor unit 16 outputs the measurement results to the CPU 11 every predetermined cycle (for example, every second, every step). The sensor unit 16 may further include sensors different from those described above. The measurement results by the sensor unit 16 include, for example, running distance, running time, pace, pitch, current position, and indicator values (form indicator values) related to the respective running form indicators. The form indicator values are related to body movement information that indicates the body movement and posture of the user during exercise (running). For each of the form indicator values, an appropriate range is set in advance. The running form indicators include: a stride [meter] that is the distance traveled in one step from one ground contact to the next; a stride-to-height ratio that is a numerical value indicating the stride in relation to height; a backward tilt of trunk [°] that is a numerical value indicating the movement of the pelvis and trunk bending backward during ground contact; a vertical movement [centimeter] that is a numerical value indicating the vertical movement of the body during running; a vertical movement-to-height ratio [%] that is a numerical value indicating the vertical movement in relation to height; hip sinking [%] that is a numerical value indicating the downward movement of the hips as the knees bend after ground contact; a lateral pelvic tilt [°] that is a numerical value indicating the tilt and downward movement of the pelvis on the opposite side of the grounded leg; lifting-up of pelvis [°] that is a numerical value indicating the movement of pulling back the pelvis that has been tilted toward the swung leg side from the ground contact to the kickout; a pelvic rotation [°] that is a numerical value indicating the rotational movement of the pelvis; pelvic rotation timing that is a numerical value indicating the gap between the ground contact timing and the timing of pelvic rotation; impact in lateral direction [m/s²] that is a numerical value indicating the magnitude of impact in the lateral (left-right) direction of the body; kickout time [ms] that is the time between when the body sinks after ground contact and when the foot leaves the ground; ground contact time [ms] that is the time between when the foot grounds and when the foot kicks out and leaves the ground; ground contact time rate [%] that is a numerical value indicating the percentage of ground contact time in one cycle (the time between one ground contact and the next ground contact); landing impact [m/s²] that is a numerical value indicating the magnitude of impact on the body immediately after ground contact; kickout acceleration [m/s²] that is a numerical value indicating the magnitude of acceleration in kicking out the ground; speed reduction amount [m/s] that is a numerical value indicating the amount of braking after ground contact; and stiffness [kN/m·kg] that is a numerical value indicating the stiffness of the spring when the entire leg is regarded as a spring, for example.

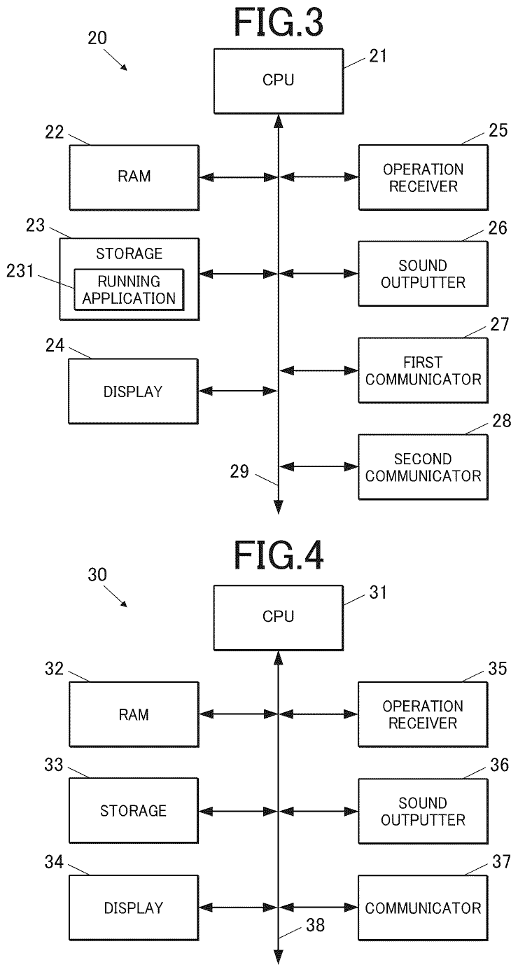

Next, the functional configuration of the information terminal 20 is explained with reference to FIG. 3. As shown in FIG. 3, the information terminal 20 is a smartphone, for example, and includes a CPU 21 (at least one processor), a RAM 22, a storage 23, a display 24, an operation receiver 25, a sound outputter 26, a first communicator 27, and a second communicator 28. These parts of the information terminal 20 are connected via a bus 29. The CPU 21 controls each part of the information terminal 20. The CPU 21 reads the designated program from system programs and application programs stored in the storage 23, loads the program in the RAM 22, and executes various processes in cooperation with the program. The CPU 21 also includes a non-illustrated timekeeping circuit and acquires the current time as timed by this timekeeping circuit. The RAM 22 is a volatile memory and forms a work area for temporarily storing various data and programs.

The storage 23 is a non-transitory computer readable medium and includes a flash memory and/or an EEPROM, for example. The storage 23 stores system programs and application programs (e.g., a running application 231) executed by the CPU 21, as well as data necessary for the execution of these programs. The running application 231 is for recording running data of the user during running, such as the running track, running distance, running time, pace, pitch, and the form indicator values described above. The running data is sequentially acquired from the sensor device 10 during running. The running application 231 has a running support mode that can provide the user with advice for improving his/her running form during running. The storage 23 also stores user information on users of the running application 231. The user information includes, for example, the user ID that is information for identifying the user, age, level of running (e.g., beginner, intermediate, advanced), sex, height, and weight of the user.

The display 24 consists of a liquid crystal display (LCD), for example, and displays screens in accordance with display control signals from the CPU 21. On the display screen of the display 24, touch sensors are provided, so that the display 24 functions as a touchscreen operation-and-display means. The operation receiver 25 includes pushbutton switches and the touch sensors disposed on the display 24. The operation receiver 25 receives input operations by the user, converts the contents of the operations into electric signals, and outputs the signals to the CPU 21. The sound outputter 26 includes a DA converter, an amplifier, and a speaker. When outputting sound, the sound outputter 26 converts sound data into analog sound data and outputs the analog sound data from the speaker. The first communicator 27 transmits and receives information to and from the server 40 via the communication network N by communication operations in accordance with a predefined communication standard. The second communicator 28 performs wireless data communication with the communicator 18 of the sensor device 10 and the communicator 37 of the earphones 30 described below, in accordance with the communication standards related to wireless communication by BLE or Wi-Fi.

Next, the functional configuration of the earphones 30 is explained with reference to FIG. 4. As shown in FIG. 4, the earphones 30 include a CPU 31 (at least one processor), a RAM 32, a storage 33, a display 34, an operation receiver 35, a sound outputter 36, and a communicator 37. These parts of the earphones 30 are connected via a bus 38. The CPU 31 controls each part of the earphones 30. The CPU 31 reads the designated program from system programs and application programs stored in the storage 33, loads the program in the RAM 32, and executes various processes in cooperation with the program. The RAM 32 is a volatile memory and forms a work area that temporarily stores various data and programs. The storage 33 is a non-transitory computer readable medium that includes a flash memory and/or an EEPROM, for example. The storage 33 stores system programs and application programs executed by the CPU 31, as well as data necessary for the execution of these programs.

The display 34 includes LED lamps and can display the ON/OFF status of the power supply, the remaining battery level, and the like. The operation receiver 35 includes a power button (not shown) to turn the power on and off and an adjustment button (not shown) to adjust the sound level to be outputted from the sound outputter 36. The CPU 31 controls each part based on the instructions from the operation receiver 35. The sound outputter 36 includes a DA converter, an amplifier, and a speaker. When outputting sound, the sound outputter 36 converts sound data into analog sound data and outputs the analog sound data from the speaker. The communicator 37 performs wireless data communication with the second communicator 28 of the information terminal 20 in accordance with the communication standards related to wireless communication by BLE or Wi-Fi.

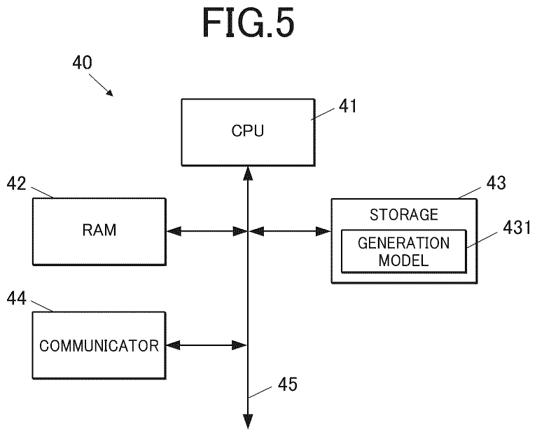

Next, the functional configuration of the server 40 is explained with reference to FIG. 5. As shown in FIG. 5, the server 40 includes a CPU 41, a RAM 42, a storage 43, and a communicator 44. These parts of the server 40 are connected via a bus 45. The server 40 may further include an operation receiver and a display to be used by the administrator of the server 40. The CPU 41 controls each part of the server 40. The CPU 41 reads the designated program from system programs and application programs stored in the storage 43, loads the program in the RAM 42, and executes various processes in cooperation with the program. The RAM 42 is a volatile memory and forms a work area that temporarily stores various data and programs. The communicator 44 transmits and receives information to and from external devices, such as the information terminal 20, via a communication network N by communication operations in accordance with a predefined communication standard.

The storage 43 is a non-transitory recording medium readable by the CPU 41 and stores various data in addition to the system programs and application programs. The storage 43 also stores a generation model 431. The generation model 431 is a text-generating AI (Artificial Intelligence) as a trained language model. The CPU 41 acquires and outputs predetermined output information by inputting certain input information to the generation model 431. Specifically, the CPU 41 inputs text data (input information) that instructs generation of text data of advice information for improving a certain running form indicator to the generation model 431 and acquires the text data of advice information (output information) generated by the generation model 431.

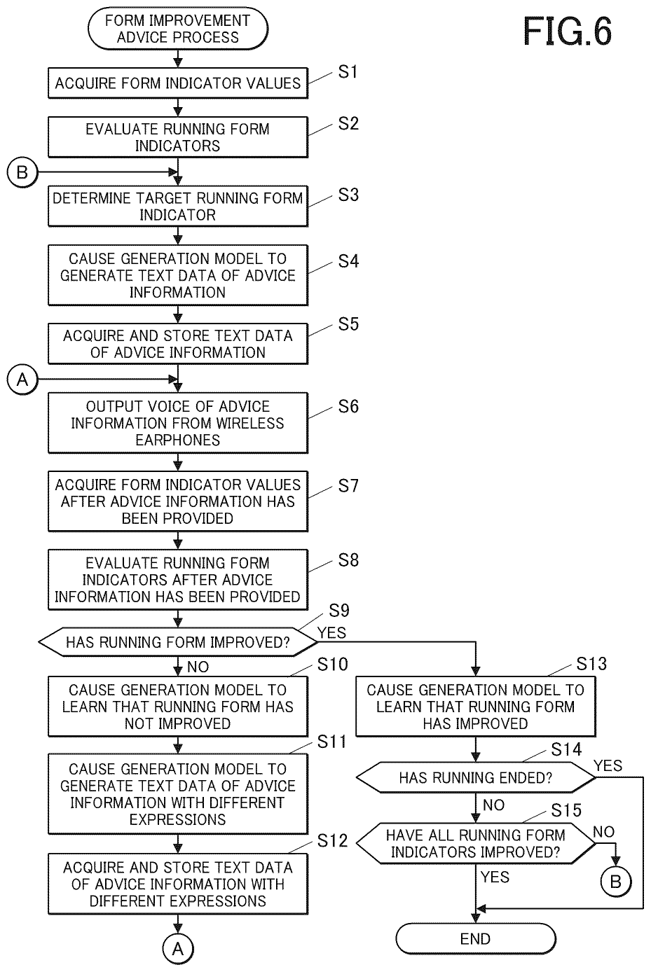

Next, the operation of the exercise support system 1 is explained. The CPU 21 of the information terminal 20 executes the form improvement advice process shown in FIG. 6 when the running support mode is selected in the running application 231, for example. When the running support mode is selected, the CPU 21 of the information terminal 20 instructs the target user, to whom the advice information for improving the running form is provided, to start running. Specifically, the CPU 21 transmits, to the earphones 30 via the second communicator 28, voice data to instruct the target user to start running. The CPU 21 then causes the sound outputter 36 of the earphones 30 to output a voice based on the voice data, such as “Please start running to grasp your current running form.”, for example.

When the form improvement advice process is started, first, the CPU 21 of the information terminal 20 receives running data measured by the sensor device 10 (sensing data from various sensors) from the sensor device 10. The CPU 21 performs arithmetic operations with the acquired running data to acquire the form indicator values of the target user (Step S1). In this embodiment, the CPU 21 acquires the above-described multiple kinds of form indicator values of the target user. In other words, the CPU 21 acquires the indicator values (form indicator values) related to body movement information of the user during exercise. The CPU 21 functions as an acquisition unit. Step S1 is the acquisition step. The information terminal 20 may transmit the running data to the server 40 via the first communicator 27, and the server 40 may calculate the form indicator values on behalf of the information terminal 20 and transmit the data of the form indicator values to the information terminal 20 via the communicator 44.

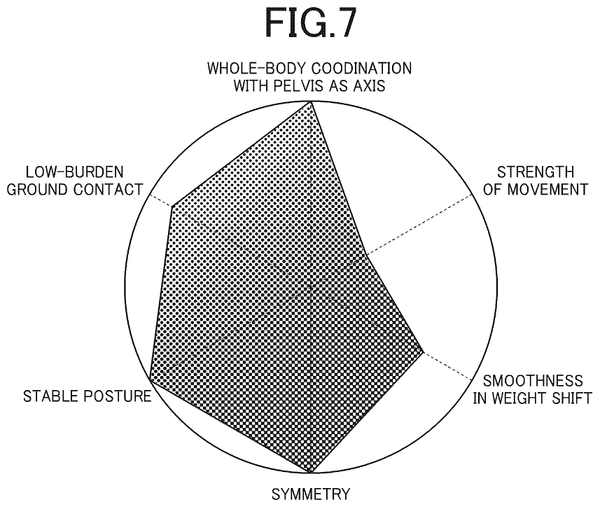

Next, the CPU 21 evaluates the running form indicators of the target user during running, based on the form indicator values acquired in Step S1 (Step S2). In Step S2, the CPU 21 scores multiple form items related to the movement and posture of the target user during running, based on the form indicator values of the target user during running, for example. Thus, the CPU 21 obtains scoring results (first scores) of the multiple form items. The information terminal 20 may transmit data of the form indicator values to the server 40 via the first communicator 27, and the server 40 may score the form items on behalf of the information terminal 20 and transmit data of scoring results of the form items to the information terminal 20 via the communicator 44. For another example, the server 40 may acquire running data from the information terminal 20, calculate the form indicator values from the running data, score the form items based on the form indicator values, and transmit data of scoring results of the form items to the information terminal 20. FIG. 7 shows an example of a radar chart showing the calculation results of the first scores for the respective form items. The form items include whole-body coordination with the pelvis as the axis, strength of movement, smoothness in weight shift, symmetry, stable posture, and low-burden ground contact, for example.

The value of “whole-body coordination with pelvis as axis” indicates how well the user moves and uses each part of the body in a balanced manner. The value of “strength of movement” indicates whether the user moves his/her body to extend the stride to make a powerful run. The value of “smoothness in weight shift” indicates whether the user restrains lateral swaying and braking to move forward efficiently. The value of “low-burden ground contact” indicates whether the user keeps restraining the load on the hips to reduce strain caused by impact. The value of “stable posture” indicates whether there is unstable pelvic movement to reduce strain on muscles, joints, and so forth. The value for “symmetry” indicates whether the user runs in a balanced manner with no difference in features between the right and left sides. For each form item, multiple running form indicators related to the form item (running form indicators to be used in scoring the form item) and the calculation formula for calculating the value of the form item from the form indicator values of the related running form indicators are determined beforehand and stored in the storage 23, for example. The CPU 21 scores each form item by using the stored calculation formula and thereby calculates the first score for each form item. For example, the running form indicators related to the form item “whole-body coordination with pelvis as axis” are “hip sinking”, “pelvic rotation timing”, and “kickout time”. The running form indicators related to the form item “strength of movement” are “stride-to-height ratio”, “lifting-up of pelvis”, and “pelvic rotation”. The running form indicators related to the form item “smoothness in weight shift” are “impact in lateral direction” and “speed reduction amount”. The running form indicators related to the form item “low-burden ground contact” are “vertical movement-to-height ratio”, “landing impact”, and “kickout acceleration”. The running form indicators related to the form item “stable posture” are “backward tilt of trunk” and “lateral pelvic tilt”.

Next, the CPU 21 determines a target running form indicator from among the multiple running form indicators (Step S3). With respect to the target running form indicator, necessary advice information is provided to the user. For example, in Step S3, the CPU 21 identifies the running form indicators related to the form item having the lowest value, based on the first scores of the respective form items calculated in Step S2. Next, among the identified running form indicators, the CPU 21 determines the running form indicator corresponding to the form indicator value that deviates most greatly from the appropriate range as the target running form indicator, for example. Instead of the information terminal 20, the server 40 may determine the target running form indicator for which the advice information is provided, based on the running data or the data of form indicator values received from the information terminal 20.

Next, the CPU 21 transmits advice generation instruction information to the server 40. The advice generation instruction information includes instructions to generate text data of advice information for improving the target running form indicator determined in Step S3 and corresponding to the user information of the target user. Thus, the CPU 21 causes the generation model 431 to generate text data of advice information for improving the target running form indicator (Step S4). The advice generation instruction information includes the user information of the target user and the form indicator value of the target running form indicator. For example, when the target user is at the beginner level in running or when the age of the target user is low, the advice generation instruction information includes instructions to generate text data of advice information without using difficult words or technical terms (e.g., pelvis) that are difficult to understand for the target user. When the CPU 41 of the server 40 receives the advice generation instruction information from the information terminal 20, the CPU 41 inputs the received advice generation instruction information into the generation model 431 and transmits text data of advice information generated by the generation model 431 to the information terminal 20. In other words, the CPU 21 generates text data of advice information by inputting input information including the form indicator value to the trained text generation model (generation model 431). Step S4 is the generation step. For example, when the target running form indicator is the vertical movement, the advice information generated in Step S4 includes “the vertical movement can be divided into two phases: one is when the foot is on the ground and the other is when the foot is in the air. When the foot is on the ground, it is important to support the body with the hips and the backs of the thighs to prevent the hips from sinking. Keep your hips high and your back straight. When the foot leaves the ground, it is important not to bounce upward. Keep a forward tilting posture and stare at a single point in front”, for example. The server 40 may determine the target running form indicator, based on the running data or the data of the form indicator values received from the information terminal 20, and generate text data of advice information for improving the target running form indicator with the generation model 431, based on the form indicator value of the target running form indicator.

When the CPU 21 of the information terminal 20 receives the text data of advice information for improving the target running form indicator from the server 40, the CPU 21 stores the received text data of advice information in the storage 23 (Step S5). The CPU 21 generates voice data for outputting voice of the text data of advice information for improving the target running form indicator received in Step S5 and transmits the voice data to the earphones 30. The CPU 21 outputs the voice based on the voice data from the sound outputter 36 of the earphones 30. Thus, the CPU 21 provides the user with the advice information for improving the target running form indicator (Step S6). In other words, the CPU 21 causes the outputter (sound outputter 36) to output the advice information on the exercise of the user, based on the form indicator value acquired in the acquisition step. The CPU 21 functions as an output controller. Step S6 is the output control step. When the user understands the advice information output from the earphones 30, the user can improve his/her running form, based on the advice information.

Next, from the sensor device 10, the CPU 21 of the information terminal 20 receives the running data measured by the sensor device 10 after the advice information has been provided to the target user. Thus, the CPU 21 acquires the form indicator value after the advice information has been provided to the target user (Step S7). Based on the form indicator value after the advice information has been provided to the target user acquired in Step S7, the CPU 21 evaluates the running form indicator after the advice information has been provided to the target user (Step S8). For example, in Step S8, the CPU 21 scores the form items of the target user during running, based on the form indicator values acquired in Step S7. The CPU 21 thus calculates the scoring results (second scores) of the respective form items after the advice information has been provided to the target user.

Based on the comparison between the first scores calculated in Step S2 and the second scores calculated in Step S8, the CPU 21 determines whether the running form of the target user has sufficiently improved after the advice information has been provided in Step S6 (Step S9). For example, the CPU 21 calculates the differences between the first scores and the second scores to determine whether the running form of the target user has sufficiently improved after the advice information has been provided in Step S6. In other words, the CPU 21 determines whether the form indicator values acquired after outputting the advice information have sufficiently improved over the form indicator values acquired before outputting the advice information. The CPU 21 functions as a determination unit. Step S9 is the determination step.

When determining that the running form of the target user has not sufficiently improved (Step S9: NO), the CPU 21 transmits first learning instruction information including instructions to learn that the running form of the target user has not sufficiently improved to the server 40. Thus, the CPU 21 causes the generation model 431 to learn that the running form of the target user has not sufficiently improved (Step S10). The first learning instruction information includes the user information of the target user and the text data of advice information provided to the target user in Step S6. The CPU 41 of the server 40 receives the first learning instruction information from the information terminal 20 and inputs the received first learning instruction information into the generation model 431. Thus, the CPU 41 causes the generation model 431 to learn that the target user corresponding to the user information could not improve his/her running form by the advice information provided in Step S6. In other words, the CPU 41 causes the generation model 431 to learn that the advice information provided to the target user in Step S6 is not appropriate for the target user corresponding to the user information. Instead of the CPU 21 of the information terminal 20, the CPU 41 of the server 40 may make the determination in Step S9. In this case, when the CPU 41 determines in Step S9 that the running form of the target user has not sufficiently improved, the CPU 41 causes the generation model 431 to learn that the running form of the target user has not improved in the same manner as described above.

Next, the CPU 21 transmits, to the server 40, advice generation instruction information including instructions to generate text data of advice information that is aimed at improving the target running form indicator and that includes different expressions from the advice information generated in Step S4. Thus, the CPU 21 causes the generation model 431 to generate text data of advice information that includes different expressions from the advice information generated in Step S4 (Step S11). The CPU 41 of the server 40 receives the advice generation instruction information from the information terminal 20 and inputs the received advice generation instruction information to the generation model 431. The CPU 41 acquires text data of advice information generated by the generation model 431 and transmits the text data to the information terminal 20. Step S11 is the generation step. The text data of advice information generated in Step S11 includes different expressions from the advice information generated in Step S4. Here, “different expressions” means phrases, words, or tone that differ from the previously used phrases, words, or tone for which no improvement was observed. For example, when the target running form indicator is the vertical movement, the advice information generated by the generation model 431 in Step S11 may be: “Aren't your hips sinking too much when your foot touches the ground? Firmly support your body with your hips and backs of your thighs. Are you keeping your waist high? Imagine that you are being pulled upward by a string from the top of your head. Aren't you bouncing upward when your foot leaves the ground? Keep a forward tilting posture. Stare at one point and be aware that your field of vision is not moving up and down. Relax and run to the end. Keep it up.” When the CPU 41 of the server 40 makes the determination in Step S9 instead of the CPU 21 of the information terminal 20, determines that the running form of the target user has not sufficiently improved, and determines that it is necessary to generate text data of advice information including different expressions from the advice information generated in Step S4 for improving the target running form indicator, the CPU 41 may generate the advice information in Step S11.

The CPU 21 of the information terminal 20 receives the text data of advice information for improving the target running form indicator from the server 40 and stores the received text data of advice information in the storage 23 (Step S12). The CPU 21 then proceeds to Step S6 in the form improvement advice process and causes the sound outputter 36 of the earphones 30 to output the voice of the advice information for improving the target running form indicator generated in Step S11. In other words, when determining in the determination step that the form indicator value has not improved, the CPU 21 causes the outputter (sound outputter 36) to output the advice information that includes different expressions from the advice information having already been outputted by the outputter and having failed to improve the form indicator value, based on the form indicator values. As described above, the CPU 21 causes the generation model 431 to generate text data of advice information that is aimed at improving the target running form indicator and that includes different expressions from the previously generated advice information, and outputs the voice of the newly generated advice information from the sound outputter 36 of the earphones 30, until the running form of the target user improves. When determining that the running form of the user has not improved (Step S9: NO), the CPU 21 may output a voice message that cheers up and encourages the user from the sound outputter 36 of the earphones 30. Such an encouraging message may be, for example, “The form may not improve overnight. Lack of muscle strength or flexibility may also be a cause, so don't be swayed by today's results. Let's work toward improvement.”

On the other hand, when determining that the running form of the target user has improved (Step S9: YES), the CPU 21 transmits second learning instruction information including instructions to learn that the running form of the target user has improved to the server 40. Thus, the CPU 21 causes the generation model 431 to learn that the running form of the target user has improved (Step S13). The second learning instruction information includes the user information of the target user and the text data of advice information provided to the target user in Step S6. The CPU 41 of the server 40 receives the second learning instruction information from the information terminal 20 and inputs the received second learning instruction information into the generation model 431. Thus, the generation model 431 learns that the target user corresponding to the user information improved the running form by the advice information provided in Step S6. In other words, the CPU 41 causes the generation model 431 to learn that the advice information provided to the target user in Step S6 is appropriate for the target user corresponding to the user information. As described above, by learning whether the user has improved the running form during running, the generation model 431 can improve accuracy in generating text data of advice information that is easily understood by the user. Thus, the generation model 431 can generate text data of advice information customized for each user. When determining that the running form of the target user has improved (Step S9: YES), the CPU 21 may output a voice message that cheers up the user from the sound outputter 36 of the earphones 30. The cheer-up message includes information that the form indicator value of the target running form indicator has improved and may be, for example: “You are so amazing. Your vertical movement is almost perfect. Stay focused and be mindful of it every day. Keep it up.”

Next, the CPU 21 determines whether running has ended (Step S14). For example, when the user presses the stop button for giving an instruction to stop running in the running application 231, the CPU 21 determines that running has ended. When determining that running has not ended (Step S14: NO), the CPU 21 determines whether all the running form indicators of the target user have improved (Step S15). When there is any running form indicator that has not improved (Step S15: NO), the CPU 21 proceeds to Step S3 in the form improvement advice process and determines the next target running form indicator. As described above, in the form improvement advice process, based on multiple kinds of form indicator values, the CPU 21 determines the priority order of the form indicator values (Step S3). The CPU 21 causes the outputter (sound outputter 36) to output advice information for each form indicator value, based on the determined priority order of the form indicator values. When determining that running has ended (Step S14: YES) or when all the running form indicators of the target user have improved (Step S15: YES), the CPU 21 ends the form improvement advice process.

In Step S3 of the above form improvement advice process, the CPU 41 of the server 40 may determine the target running form indicator instead of the CPU 21 of the information terminal 20. In this case, in Steps S10 and S13 of the form improvement advice process, the generation model 431 has already learned user information of multiple users and statistical information including determination results indicating whether the running forms of the users have improved by the provided advice information. Instead of Step S3, the CPU 41 inputs the user information of the target user, each form indicator value, and an instruction to determine the target running form indicator to the generation model 431. Thus, the CPU 41 causes the generation model 431 to output the target running form indicator that is expected to be easily improved by the target user. For example, the CPU 41 causes the generation model 431 to determine the target running form indicator, based on the level of running of the target user.

As described above, the exercise support method of the present embodiment includes: the acquisition step (Step S1) of acquiring an indicator value (form indicator value) related to body movement information of the user during exercise; the output control step (Step S6) of outputting advice information on exercise of the user from the outputter (sound outputter 36), based on the indicator value acquired in the acquisition step; and the determination step (Step S9) of determining whether the indicator value acquired after outputting the advice information has improved over the indicator value acquired before outputting the advice information. When it is determined in the determination step that the indicator value has not improved even though the outputter outputted the advice information, in the output control step, the outputter is caused to output advice information that includes different expressions from the advice information that has already been outputted and with which the indicator value has not improved. Therefore, when the running form of the user has not improved, the exercise support method can present the user with advice information including such expressions that help the user improve his/her running form. Such a method efficiently improves the indicator values related to body movement information of the user during exercise. Since the information terminal 20 narrows down the target form indicator from multiple form items, the server 40 only needs to input text data that instructs generation of text data of advice information for improving only the target form indicator into the generation model 431. Therefore, the server 40 does not need to input text data that instructs generation of text data of advice information for improving the form indicators that do not require advice information, other than the target form indicator. Thus, the server 40 can quickly generate text data of advice information while reducing the load on the CPU 41, and the target user can quickly receive advice during exercise.

In the exercise support method of the present embodiment, in the output control step, the outputter (sound outputter 36) is caused to output the advice information corresponding to at least one of the age and the level of exercise of the user. Thus, the user can be provided with appropriate advice information corresponding to at least one of the age and the level of exercise of the user.

In the exercise support method of the present embodiment, in the acquisition step, multiple kinds of indicator values (form indicator values) can be acquired. Based on the multiple kinds of indicator values, in the output control step, the priority order of the indicator values is determined; and the outputter (sound outputter 36) is caused to output the advice information for each indicator value according to the priority order. Thus, the advice information for improving the running form indicator having the form indicator value that deviates most greatly from the appropriate range can be preferentially presented to the user. This efficiently improves the exercise form of the user.

In the exercise support method of the present embodiment, the indicator value (form indicator value) is related to a predetermined form indicator (running form indicator) of the body movement of the user during exercise. The indicator value is measured by the motion sensor (sensor device 10) attached to the user's body. With the sensor device 10 attached to the user's body, the form indicator values can be easily obtained.

The exercise support method further includes the generation step (Steps S4, S11). In the generation step, input information including the indicator value (form indicator value) is input to the trained text generation model (generation model 431) to generate text data of advice information. Thus, the text data of advice information for improving the target running form indicator can be easily generated.

The above-described embodiment is an example of the exercise support method, the exercise support device, the exercise support system, and the non-transitory computer readable medium according to the present disclosure. The embodiment is not intended to limit the present disclosure. For example, the target running form indicator in the form improvement advice process may be set based on the user operation in the running application 231 launched on the information terminal 20. In the above embodiment, the advice generation instruction information may include information such as the target user's location during running, running time, running distance, environmental conditions (e.g., date and time, weather conditions such as weather, temperature, humidity, wind force, wind direction, and rainfall), running path conditions (e.g., incline), heart rate, and fatigue level. In this case, the CPU 41 generates text data of advice information according to the above information by the generation model 431 and transmits the text data to the information terminal 20. The CPU 21 provides the target user with the advice information according to the above information by voice from the earphones 30.

In the above embodiment, the exercise support system 1 includes the sensor device 10, the information terminal 20, the earphones 30, and the server 40. However, the sensor device 10 and the information terminal 20 need not be separate but may be an integrated device. Further, part of the functions of the information terminal 20 may be provided to the server 40. For example, at least one of the following functions may be provided to the server 40: the function to acquire the indicator values related to body movement information of the user during exercise; the function to cause the outputter to output advice information on the exercise of the user based on the acquired indicator values; the function to determine whether the indicator value acquired after outputting the advice information has improved over the indicator value acquired before outputting the advice information; and the function to cause the outputter to output advice information that includes different expressions from the advice information that has already been outputted and that has not improved the indicator value when it is determined in the determination step that the indicator value has not improved after the advice information has been outputted from the outputter. Instead of the information terminal 20, a communication terminal capable of communicating with the server 40 and the sensor device 10 may be used. Thus, the exercise support device is not limited to the information terminal 20 but may be composed of at least one of the sensor device 10, the information terminal 20, and the server 40. In the above embodiment, the outputter is the sound outputter 36. The outputter may be at least either the display 14 of the sensor device 10 or the display 24 of the information terminal 20. In this case, at least one of the sound outputter 17 of the sensor device 10, the sound outputter 26 of the information terminal 20, and the sound outputter 36 of the earphones 30 should output an alert sound or voice to inform that advice texts are displayed on the display 14 or the display 24 of the information terminal 20. In the above embodiment, advice information is provided to the user regarding running as the exercise performed by the user. However, the present invention is not limited to this. The exercise other than running may be the target of the advice information. Further, the computer-readable medium that stores the program according to the present invention can be an information recording medium, such as a CD-ROM. Further, a carrier wave is also applicable to the present invention as a medium for providing data of the program according to the present invention via communication lines. The detailed configuration and detailed operation of each component of the sensor device 10, the information terminal 20, the earphones 30, and the server 40 in the above embodiment can be changed as appropriate without departing from the scope of the present invention.

1. An exercise support method comprising:
acquiring an indicator value related to body movement information of a user during exercise;
causing an outputter to output advice information on the exercise of the user, based on the acquired indicator value; and
determining whether the indicator value acquired after outputting the advice information has improved over the indicator value acquired before outputting the advice information, wherein
in a case where it is determined that the indicator value has not improved even though the outputter has outputted the advice information, the outputter is caused to output advice information that includes a different expression from the advice information that has already been outputted and with which the indicator value has not improved.

2. The exercise support method according to claim 1, wherein the outputter is caused to output the advice information corresponding to at least one of an age of the user and a level of the exercise of the user.

3. The exercise support method according to claim 1, wherein multiple kinds of indicator values each of which is the indicator value are acquired.

4. The exercise support method according to claim 3, wherein:
based on the multiple kinds of indicator values, a priority order of the indicator values is determined, and
based on the priority order, the outputter is caused to output the advice information for each of the indicator values.

5. The exercise support method according to claim 1, wherein the indicator value is related to a predetermined form indicator of a body movement of the user during the exercise and is measured by a sensor attached to a body of the user.

6. The exercise support method according to claim 1, further comprising inputting input information that includes the indicator value into a trained text generation model to generate text data of the advice information.

7. An exercise support device comprising at least one processor, wherein the at least one processor:
acquires an indicator value related to body movement information of a user during exercise,
causes an outputter to output advice information on the exercise of the user, based on the acquired indicator value, and
determines whether the indicator value acquired after outputting the advice information has improved over the indicator value acquired before outputting the advice information,
wherein in response to determining that the indicator value has not improved even though the outputter has outputted the advice information, the at least one processor causes the outputter to output advice information that includes a different expression from the advice information that has already been outputted and with which the indicator value has not improved.

8. The exercise support device according to claim 7, wherein the at least one processor causes the outputter to output the advice information corresponding to at least one of an age of the user and a level of the exercise of the user.

9. The exercise support device according to claim 7, wherein the at least one processor acquires multiple kinds of indicator values each of which is the indicator value.

10. The exercise support device according to claim 9, wherein:
based on the multiple kinds of indicator values, the at least one processor determines a priority order of the indicator values, and
based on the priority order, the at least one processor causes the outputter to output the advice information for each of the indicator values.

11. The exercise support device according to claim 7, wherein the indicator value is related to a predetermined form indicator of a body movement of the user during the exercise and is measured by a sensor attached to a body of the user.

12. The exercise support device according to claim 7, wherein the at least one processor inputs input information that includes the indicator value into a trained text generation model to generate text data of the advice information.

13. An exercise support system comprising:
a sensor device;
an information terminal; and
a server, wherein:
based on data related to body movement information of a user during exercise and acquired from the sensor device, the information terminal acquires an indicator value related to the body movement information,
based on the acquired indicator value, the information terminal or the server scores form items related to the exercise of the user,
the information terminal or the server determines a target form indicator from the form items,
in response to a request from the information terminal, the server generates advice information for improving the determined target form indicator and transmits the generated advice information to the information terminal, and
the information terminal causes an outputter to output the advice information.

14. The exercise support system according to claim 13, wherein the advice information is generated, based on at least one of an age of the user and a level of the exercise of the user.

15. The exercise support system according to claim 13, wherein the server inputs input information that includes the indicator value into a trained text generation model to generate text data of the advice information.

16. A non-transitory computer readable storage medium storing a program to be read and executed by a computer of an exercise support device, the program causing the computer to:
acquire an indicator value related to body movement information of a user during exercise,
cause an outputter to output advice information on the exercise of the user, based on the acquired indicator value, and
determine whether the indicator value acquired after outputting the advice information has improved over the indicator value acquired before outputting the advice information,
wherein in response to determining that the indicator value has not improved even though the outputter has outputted the advice information, the computer causes the outputter to output advice information that includes a different expression from the advice information that has already been outputted and with which the indicator value has not improved.
